(19) United States
(12) Patent Application Publication
(10) Pub. No.: US 2026/0151246 A1
(43) Pub. Date: Jun. 4, 2026

(54) PUMP ASSEMBLY FOR AN ORTHOPEDIC DEVICE

(71) Applicant: OSSUR ICELAND EHF, Reykjavik (IS)

(72) Inventors: Larus GUNNSTEINSSON, Reykjavik (IS); Valgeir PETURSSON, Reykjavik (IS)

(21) Appl. No.: 19/122,620

(22) PCT Filed: Oct. 19, 2023
(86) PCT No.: PCT/US2023/077252

§ 371 Date: Apr. 18, 2025

Related U.S. Application Data
(60) Provisional application No. 63/418,121, filed on Oct. 21, 2022.

(51) Int. Cl. A61F 5/01

(52) U.S. Cl. CPC A61F 5/0111; A61F 5/012

(57) ABSTRACT

An orthopedic device, such as a leg walker or walking boot, includes a body formed from a resilient and air-impermeable material, the body forming an outer wall. A pump cover extends over a portion of the outer wall and forms a chamber with the outer wall. The pump cover secures to the outer wall such that the chamber is delimited by the outer wall and the pump cover. An inlet opening is defined by the outer wall and communicating with the chamber. An outlet opening is defined by the outer wall and communicates with the chamber. An airflow valve disposed of in the chamber. An outer wall forms a backing surface to the pump cover such that the chamber is only contained by the outer wall and the pump cover.

### FIELD OF THE DISCLOSURE

The disclosure relates to an orthopedic device for providing a pump assembly in an orthopedic device, such as a leg walker for protecting and/or immobilizing one or more affected areas on a user's lower leg, ankle, and/or foot.

### BACKGROUND

Sprains, fractures, and soft tissue injuries involving the lower leg and foot commonly result from household accidents, workplace incidents, and sports-related trauma. Other wounds or sensitive areas in the lower limbs may result from surgical intervention or the effect of certain medical conditions. These injuries affect a broad range of individuals and, while not life-threatening, can increase in severity without treatment, stabilization, and/or protection.

Prior art solutions for treating, stabilizing, and/or protecting the lower limb after injury or surgery can be categorized into casting systems and orthopedic braces. Each approach can provide the required rigid support to a user's limb, with distinct disadvantages and drawbacks.

Known casting systems are typically fabricated on a user's limb and conform to the unique anatomy. The casting systems comprise an interior padding and an exterior layer of materials moldable in a first state before transitioning into a rigid material state, e.g., molded plasters or resins applied to a limb and then hardened in place. The casting systems are often difficult and messy to create, are not adjustable once hardened, are not easily removed without being destroyed, are not reusable, are not breathable or hygienic, and must be worn for long, uninterrupted periods.

Orthopedic braces include various splints, braces, and walking boots. The braces can be mass-produced and form complex multi-component systems that allow adjustment or tightening of a user's limb. Such multi-component systems often include several straps or other securing means, with rigid plastic shells or splints for securing a padded structure around a limb, enclosing or wrapping the limb in a soft or padded internal covering inflatable support, with a harder frame or external shell. Unfortunately, the complexity of the multi-component systems and the cost of the required materials render orthopedic braces uneconomical for personalized construction conforming to the anatomy or treatment needs.

There is a need for a stabilizing solution adaptable to a user's anatomy at a low cost, which is adjustable about the limb of a user, with fewer or no complex multi-component systems.

Existing walkers may include inflatable supports to improve the comfort of the walker. The walker may include a liner having a bladder with a pump associated therewith for regulating the pressure of the bladder. In such conventional arrangements, a pump may be provided with the liner and bladder, including an external pump as in U.S. Pat. No. 5,078,128, granted on Jan. 7, 1992, including integrating the pump with a dorsal shell as in U.S. Pat. No. 8,506,510, granted on Aug. 8, 2013, or a strap as in U.S. Pat. No. 10,993,826, granted on May 4, 2021, each of these patents incorporated herein by their entirety. Other examples exist in the prior art whereby the pump is secured to the liner, so the pump and liner with the bladder are an integral unit.

It is desired to provide a simplified pump assembly that, unlike in the prior art, is secured or is formed in part by the frame or body of the orthopedic device or walker. From a manufacturing standpoint, the pump assembly can be simply formed with fewer components and take advantage of using the body of the orthopedic walker to be part of the pump assembly. In addition, the liner or inflatable support can be offered without the pump, thereby offering flexibility in how the liner is constructed without the constraint of a pump integrated therewith.

The pump can be associated with the inflatable support to minimize being bumped, misaligned, and/or damaged during use, increasing the likelihood that the inflatable supports will be inadvertently inflated, deflated, and/or rendered inoperable. Likewise, the pump can be formed at a location offering facile operation of the pump assembly for various patients.

### SUMMARY

A pump assembly is integrated with a wall of the orthopedic device to offer convenient and simple regulation of pressure in an inflatable support associated with the orthopedic device. The wall of the orthopedic device forms a backing surface to the pump and therefore excludes a need to provide a separate or external pump. Instead, the pump can be simply manufactured and adaptable to various inflatable supports, including tubing adapted to secure to the pump assembly.

An example of such an orthopedic device is a leg walker, although other orthopedic devices may have frames or bodies configured to accommodate a pump assembly of the disclosure arranged to couple to an inflatable support.

According to an embodiment of the orthopedic device or pump assembly, the pump assembly is defined by an outer wall formed from a resilient and air-impermeable material forming at least part of the body of the orthopedic device. A pump cover extends over a portion of the outer wall, forms a chamber, and is secured to the outer wall. The outer wall and the pump cover delimit the chamber. The outer wall defines an inlet opening and communicates with the chamber. The outer wall defines an outlet opening and communicates with the chamber to permit the extension of tubing therethrough for engaging a valve disposed of in the chamber and configured for receiving the tubing extending to an inflatable support. The outer wall forms a backing surface to the pump cover such that the chamber is only contained by the outer wall and the pump cover.

The numerous other advantages, features, and functions of embodiments of an orthopedic device having a pump assembly or a pump assembly itself having the features discussed herein will become readily apparent and better understood because of the following description and accompanying drawings. The following description is not intended to limit the scope of the orthopedic device and pump assembly but merely provides exemplary embodiments for ease of understanding.

### BRIEF DESCRIPTION OF THE DRAWINGS

These and other features, aspects, and advantages of the present invention will become better understood concerning the following description, appended claims, and accompanying drawings.

FIG. 1 is an elevational view of an orthopedic device in the form of a leg walker having a pump assembly, according to the disclosure.

FIG. 2 is a sectional plan view showing a detail of the orthopedic device of FIG. 1.

FIG. 3 is a schematic view of the inside of the pump cover with an intake valve and airflow valve in the pump assembly of FIG. 1.

FIG. 4 is a detailed sectional view of the outer wall of the body of the orthopedic device of FIG. 1 without the pump cover.

FIG. 5 is a detailed sectional of an inner wall of the body of the orthopedic device of FIG. 1.

### DETAILED DESCRIPTION OF VARIOUS EMBODIMENTS

A better understanding of different embodiments of the disclosure may be had from the following description read with the accompanying drawings in which reference characters refer to like elements.

While the disclosure is susceptible to various modifications and alternative constructions, certain illustrative embodiments are described in the drawings below. It should be understood, however, that there is no intention to limit the disclosure to the embodiments disclosed, but on the contrary, the intention covers all modifications, alternative constructions, combinations, and equivalents falling with the spirit and scope of the disclosure.

An example of an orthopedic device is in U.S. patent application publication No. 2019/0240057, published on Aug. 8, 2019, and U.S. patent application publication no. 2022/0151812, published on May 19, 2022, each of the aforementioned patent application publications incorporated herein by reference. Exemplary inflatable supports are found in U.S. Pat. Nos. 5,078,128, 8,506,510, 10,993,826, or U.S. patent application publication No. 2022/0151812. Any inflatable support with a connection adapted to receive a pump assembly may be adapted for the following embodiments of an orthopedic device having a pump assembly, a pump assembly, and a method for manufacturing or operating a pump assembly.

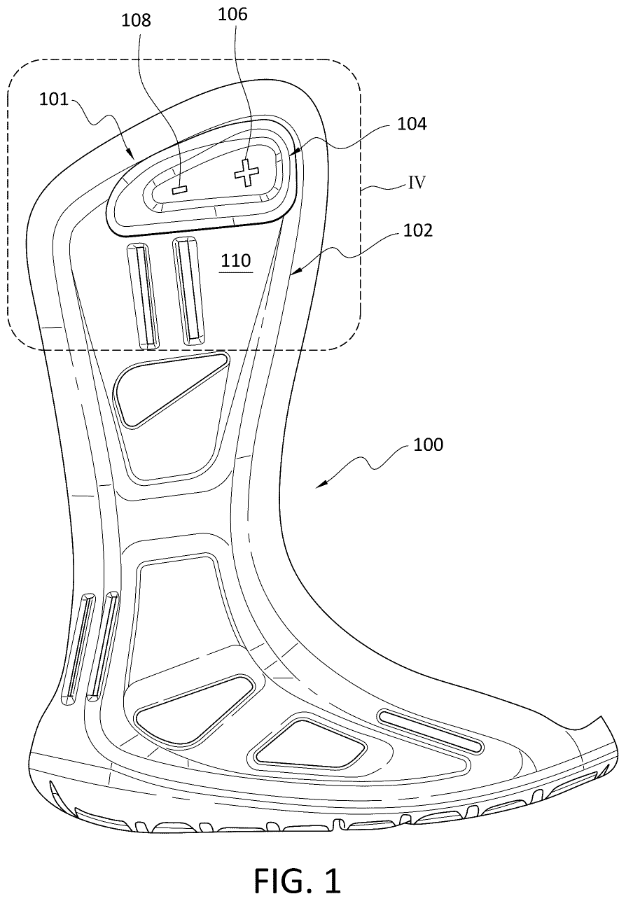

FIG. 1 depicts an orthopedic walker having an orthopedic device 100 defining an outer wall or outer wall 110 formed from a resilient and air-impermeable material forming at least part of a body 102 of the orthopedic device 100. A pump assembly 101 is integrated with the outer wall 110 of the orthopedic device 100 to offer convenient and simple regulation of pressure associated with the orthopedic device 100. As used herein, the term “integrated” means that two or more components have a homogeneous or continuous connection. The pump assembly 101 comprises a pump cover 104 arranged to extend over and secure to a portion of the outer wall 110. A chamber 105 is formed and delimited by the outer wall 110, and the pump cover 104. By having the chamber 105 formed and delimited by the outer wall 110, the size and contour of the pump cover 104 may be reduced to minimize the profile of the pump assembly 101. An inlet opening 118 is defined by the outer wall 110, and communicates with the chamber 105. An outlet opening 120 is defined by the outer wall 110, and communicates with the chamber 105. An airflow valve 124 is disposed in the chamber 105 to regulate airflow therethrough and adapted to secure tubing 121 extending to an inflatable support.

In a preferred embodiment, the outer wall 110 is formed by ethyl-acetate vinyl (EVA) or similar material and is molded and constructed to be air-impermeable. Indeed, the outer wall 110 may be constructed solely by EVA, thereby consisting EVA. For example, at least at the location 113 of the outer wall 110 at which the pump cover 104 secures, the outer wall 110 is molded in a compressed or air-impermeable manner. Areas outside the receiving location 113 may be molded, formed differently, or constructed with different materials. Alternatively, the wall portion may be formed from a different material or coated by a material at the location to ensure that it is air-impermeable.

While EVA is mentioned as a preferred material in U.S. patent application publication no. 2022/0151812, other materials may be envisioned with either air-impermeable characteristics or molded or otherwise formed in an air-impermeable manner.

From the aforementioned construction of the pump assembly 101, the outer wall 110 forms a backing surface to the pump cover 104 such that the outer wall 110 only contains the chamber 105 and the pump cover 104. Both the outer wall 110 and the pump cover 104 may be configured, as in being molded or machined, to accommodate features of the valve, thereby minimizing components and creating a securing positioned pump assembly 101 on the body 102 of the orthopedic device 100.

The receiving location 113 is preferably configured and dimensioned to a shape corresponding to the periphery 125 of the pump cover 104. The pump cover 104 is preferably formed from a material and structurally defined as more resilient or flexible than the outer wall 110, such that when the pump cover 104 is pressed for inflation or deflation, the pump cover 104 yields to pressure and can activate the airflow valve (at least for deflation). The outer wall 110 withstands and does not yield to pressure exerted by the user. Particularly, the outer wall 110 is sufficiently rigid to not yield to pressure whether or not a user is wearing the orthopedic device.

Referring to FIGS. 1-3, the pump cover 104 is defined by an inflation portion 114 and a deflation portion 116 divided by a dividing section 128 formed by the pump cover 104. The pump cover 104 defines an indicia 106 for inflation, corresponding to the inflation portion 114. The pump cover likewise defines indicia 108, corresponding to the deflation portion 116. The indicia may be formed or molded into the pump cover or applied by printing or adhesives.

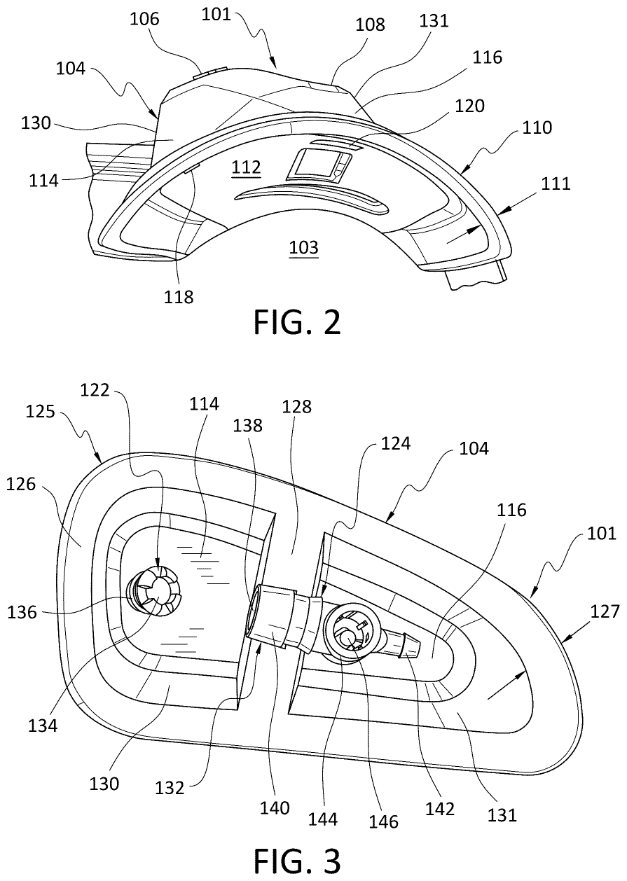

As shown in part by FIGS. 2 and 3, the inflation portion 114 has a greater depth 130 than the depth or height 131 (defined as the extent protruding from the outer wall 110) of the deflation portion 116. This is because the inflation portion 114 and the deflation portion 116 are continuously formed by the pump cover 104 without interruption and are inseparable from the outer surface of the pump cover 104, although they are impermeably separated by the dividing section 128 and the airflow valve 124.

Features of the body 102 are adapted to accommodate the pump assembly 101. The outlet opening 120 is configured and dimensioned to permit the expulsion of air therethrough by activating the airflow valve 124 and to receive and permit extension of the tubing 121 into the pump assembly 101 therethrough for coupling to a bladder or reservoir of the inflatable support.

As exemplified by FIGS. 2-5, a fill valve 122 is secured in a wall thickness 111 of the body 102 and communicates a body cavity 103 or ambient air from an inner wall 112 of the walker body 102 to the chamber 105. The fill valve 122 preferably defines a threaded insert 136 secured into the wall thickness 111 and inlet 134 for drawing air into chamber 105.

The airflow valve 124 defines an inlet 138 extending into the chamber 105 of the inflation portion 114, a release valve 144 having a plunger 146 arranged to be located in the deflation portion 116, and a connector 142 adapted to secure to tubing (not shown) extending to a bladder or other reservoir for offering inflation means in the orthopedic device and pressurization of the bladder or other reservoir. The pump assembly 101 function is such that upon pressing the inflation portion 114, air is drawn through the fill valve 122 into the chamber 105 and through the airflow valve 124 to the connector 142; upon pressing the deflation portion 116, the plunger 146 is depressed to depressurize the bladder or reservoir.

The pump cover 104 defines a mating edge surface 126 adapted to be secured to the outer wall 110 of the orthopedic device 100. The mating edge surface 126 is secured to the outer wall 110 by an adhesive not shown that is air impermeable. The mating edge surface 126 has a width 127, providing a sufficient surface area to fixedly secure along the outer wall 110 in a flush manner, such that the width 127 is parallel continuously along the outer wall 110 and acts as an interface to seal against the outer wall 110. In addition, the mating edge surface 126 defines the same width and continuous surface area to ensure consistent and secure sealing against the outer wall 110. In an embodiment, the mating edge surface 126 is secured to the outer wall 110 of the orthopedic device 100 by one or more fastening means, such as a clip, buckle, or screw, that is sufficient to create an airtight or air-impermeable seal.

The mating edge surface 126 defines a dividing section 128 for separating the chamber 105 of the inflation portion 114 and the deflation portion 116. The mating edge surface 126 secures against the outer wall 110 at the dividing section so that the inflation portion 114 and the deflation portion 116 are impermeably separated aside from the airflow valve 124. The dividing section 128 forms a recess 132 configured and dimensioned to receive a periphery 140 of the inlet 134. The airflow valve 124 is impermeably secured by the dividing section 128 between the inflation portion 114 and the deflation portion 116.

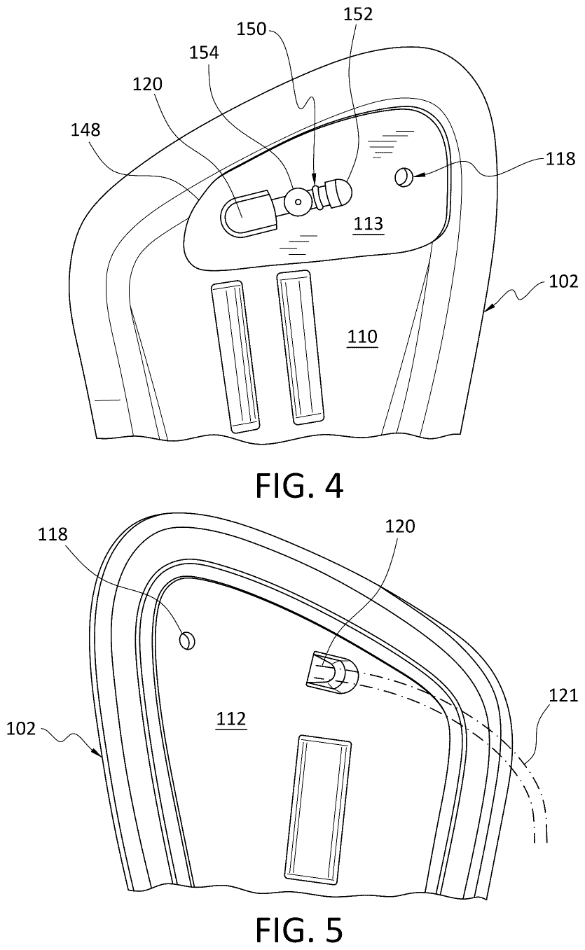

As shown in FIG. 4, the outer wall 110 may define a recess 148 configured and dimensioned to receive the periphery 125 of the pump cover 104. The mating edge surface 126 fits entirely within the recess 148. The recess 148 may form a wall thickness such that the mating edge surface 126 is flush with the surface of the outer wall, and another peripheral edge of the pump cover 104 is flush with the wall thickness of the recess to assure multiple areas of adhesion and sealing of the pump cover 104 to the outer wall 110.

As illustrated in FIG. 4, the outer wall 110 forms a seated portion 150 defined to correspond to portions of the airflow valve 124. The seated portion 150 preferably defines a first portion 152 adapted to receive the periphery 140 of the inlet 134, and a second portion 154 adapted to receive at least a portion of the release valve 144.

According to the aforementioned embodiments, the disclosure envisions a manufacturing method or methods for assembling the pump assembly according to the aforementioned features. Likewise, the disclosure embraces methods for using the pump assembly according to the preceding discussion.

While various aspects and embodiments have been disclosed herein, other aspects and embodiments are contemplated. The various aspects and embodiments disclosed herein are for illustration purposes and are not intended to be limiting. Additionally, the words “including,” “having,” and variants thereof (e.g., “includes” and “has”) as used herein, including the claims, shall be open-ended and have the same meaning as the word “comprising” and variants thereof (e.g., “comprise” and “comprises”).

It is to be understood that not necessarily all objects or advantages may be achieved under any embodiment of the disclosure. Those skilled in the art will recognize that the orthopedic device and pump assembly may be embodied or carried out in a manner that achieves or optimizes one advantage or group of advantages as taught herein without necessarily achieving other objects or advantages as may be taught or suggested herein.

The skilled artisan will recognize the interchangeability of various disclosed features. Besides the variations described herein, other known equivalents for each feature can be mixed and matched by one of ordinary skill in this art to construct an orthopedic device and pump assembly in accordance with principles of the present disclosure. Further, it will be understood by the skilled artisan that the features described herein may be adapted to other types of devices. Hence this disclosure and the embodiments and variations thereof are not limited to pump assemblies for an orthopedic device, such as a leg walker, but can be utilized in any device.

Although this disclosure describes certain exemplary embodiments and examples of an orthopedic device and pump assembly, it will be understood by those skilled in the art that the present disclosure extends beyond the specifically disclosed embodiments to other alternative embodiments and/or uses of the disclosure and obvious modifications and equivalents thereof. It is intended that the present disclosure should not be limited by the disclosed embodiments described above and may be extended to other applications that may employ the features described herein.

1.-20. (canceled)

21. An orthopedic device, comprising:
a body formed from a resilient and air-impermeable material, the body forming an outer wall;
a pump cover extending over a portion of the outer wall and forming a chamber with the outer wall, the pump cover secured to the outer wall such that the chamber is delimited by the outer wall and the pump cover;
an inlet opening defined by the outer wall and communicating with the chamber;
an outlet opening defined by the outer wall and communicating with the chamber;
an airflow valve disposed in the chamber;
wherein the outer wall forms a backing surface to the pump cover such that the chamber is only contained by the outer wall and the pump cover;
wherein the outer wall is molded in a configuration in a non-porous to form an air-impermeable surface at a receiving location for receiving the pump cover;
wherein the receiving location is configured and dimensioned to a shape corresponding to a periphery of the pump cover.

22. The orthopedic device of claim 21, wherein the outlet opening is configured and dimensioned to permit expulsion of air therethrough, and to receive and permit extension of tubing extending therethrough for coupling to a bladder or reservoir.

23. The orthopedic device of claim 21, wherein the outer wall is constructed solely by ethyl-acetate vinyl (EVA).

24. The orthopedic device of claim 21, wherein areas of the outer wall outside of the receiving location are molded or formed differently from the receiving location.

25. The orthopedic device of claim 21, wherein the receiving location is formed by ethyl-acetate vinyl (EVA) and the pump cover is formed by EVA, the pump cover is secured to the outer wall along the receiving location by an adhesive which is air impermeable.

26. The orthopedic device of claim 21, wherein the pump cover is formed from a material that is structurally defined as more resilient than the outer wall.

27. The orthopedic device of claim 26, wherein the pump cover is defined by an inflation portion and a deflation portion, divided by a dividing section formed by the pump cover.

28. The orthopedic device of claim 27, wherein the pump cover defines an increase indicia corresponding to the inflation portion, and a decrease indicia corresponding to the deflation portion.

29. The orthopedic device of claim 27, wherein the valve defines an inlet extending into the chamber of the inflation portion, a release valve having a plunger located in the deflation portion, and a connector adapted to secure to tubing extending to a bladder or other reservoir for offering inflation means in the orthopedic device and pressurization of the bladder or other reservoir.

30. The orthopedic device of claim 21, further comprising a fill valve secured in a wall thickness of the body, communicating a body cavity from ambient air therein from an inner wall of the body to the chamber.

31. The orthopedic device of claim 30, wherein the fill valve defines a threaded insert secured into the wall thickness, and an inlet for drawing air therethrough into the chamber.

32. The orthopedic device of claim 21, wherein the pump cover defines a mating edge surface adapted to be secured to the outer wall of the orthopedic device.

33. The orthopedic device of claim 32, wherein the mating edge surface is secured to the outer wall by an adhesive (not shown) that is air impermeable.

34. The orthopedic device of claim 33, wherein the mating edge surface has a width providing a sufficient surface area to fixedly secure along the outer wall in a flush manner, such that the width is parallel continuously along the outer wall and acts as an interface to seal against the outer wall.

35. The orthopedic device of claim 32, wherein the mating edge surface, defines a same width and continuous surface area along its entirety to assure consistent and secure sealing against the outer wall.

36. The orthopedic device of claim 35, wherein the mating edge surface defines a dividing section for separating the chamber of an inflation portion and a deflation portion, the mating edge surface securing against the outer wall at the dividing section so that the inflation portion and the deflation portion are impermeably separated from one another aside from the airflow valve.

37. The orthopedic device of claim 36, wherein the dividing section forms a recess, configured and dimensioned to receive a periphery of the inlet, such that the airflow valve is impermeably secured by the dividing section between the inflation portion and the deflation portion.

38. The orthopedic device of claim 37, wherein the outer wall defines a recess configured and dimensioned to receive the periphery of the pump cover, such that the mating edge fits entirely within the recess.

39. The orthopedic device of claim 38, wherein the outer wall forms a seated portion defined to correspond to portions of the airflow valve, the seated portion defining a first portion adapted to receive the periphery of the inlet, and a second portion adapted to receive a release valve.

40. The orthopedic device of claim 37, wherein the inflation portion is sized larger having a greater depth than a depth or height, said depth or height defined as an extent protruding from the outer wall, of the deflation portion, the inflation portion and the deflation portion being continuously formed by the pump cover without interruption and are inseparable about an outer surface of the pump cover, although impermeably separated by the dividing section and the airflow valve.
